(19) United States
(12) Patent Application Publication
(10) Pub. No.: US 2016/0144193 A1
(43) Pub. Date: May 26, 2016

(54) MEDICAL IMPLANT WITH A COMMUNICATIONS INTERFACE

(71) Applicant: Biotronik SE & Co. KG, Berlin (DE)

(72) Inventors: Thomas DOERR, Berlin (DE); Michael DIEBOLD, Berlin (DE)

(73) Assignee: BIOTRONIK SE & CO. KG, Berlin (DE)

(21) Appl. No.: 14/880,491

(22) Filed: Oct. 12, 2015

Related U.S. Application Data
(60) Provisional application No. 62/083,899, filed on Nov. 25, 2014.

(51) Int. Cl. A61N 1/372; A61N 1/39; A61N 1/378; A61N 1/375; A61N 1/36; A61N 1/365

(52) U.S. Cl. CPC A61N 1/37241; A61N 1/36003; A61N 1/36135; A61N 1/365; A61N 1/378; A61N 1/3756; A61N 1/3987

(57) ABSTRACT

Embodiments include a medical implant that is inserted into a human and/or animal body. The medical implant includes an energy source, a pulse generator, a control unit and at least one electrode pair that delivers electrical stimulation pulses to a bodily tissue. Embodiments include a sensor, provided on an implant side of the medical implant, that identifies an implant-external trigger element. The control unit is coupled to the sensor and may be activated depending on a sensor signal of the sensor to perform an adaptation sequence of stimulation parameters, in which test stimulation pulses may be outputted. Embodiments include a method of setting a parameter set of therapeutic electrical stimulation pulses of a medical implant.

This application claims the benefit of U.S. Provisional Patent Application 62/083,899 filed on 25 Nov. 2014, the specification of which is hereby incorporated herein by reference.

### BACKGROUND OF THE INVENTION

1. Field of the Invention

Embodiments of the invention generally relate to a medical implant with a communications interface.

2. Description of the Related Art

Generally, active electronic implants for insertion into the human and/or animal body have a bidirectional communications interface for a telemetry function to be able to set stimulation parameters. Typically, the telemetry function of the active electronic implant requires an antenna that is suitable for telemetry and which is adapted to the frequency to be used. However, generally, the antenna's geometry significantly limits the possibility of miniaturizing such implants.

### BRIEF SUMMARY OF THE INVENTION

One or more embodiments of the invention provide an implant with telemetry possibilities, such that extensive miniaturization of the implant may be provided compared with known devices.

At least one embodiment of the invention is achieved in accordance with elements of the independent claims. Embodiments of the invention will emerge from the further claims, the description and the drawings as presented herein.

One or more embodiments of the invention include a medical implant that may be inserted into a human and/or animal body. In at least one embodiment, the medical implant may include one or more of an energy source, a pulse generator, a control unit and at least one electrode pair that deliver electrical stimulation pulses to a bodily tissue. One or more embodiments may include a sensor on an implant side of the medical implant that identifies an implant-external trigger element. In at least one embodiment, the control unit is coupled to the sensor and may be activated depending on a sensor signal of the sensor in order to perform an adaptation sequence of stimulation parameters, wherein test stimulation pulses may be outputted by the control unit.

By way of at least one embodiment, a determination of a stimulation stimulus threshold and subsequent programming of the stimulation energy may be performed via the adaptation sequence. One or more embodiments may include a miniaturized implant without a telemetry antenna, wherein the pulse energy may be set individually for each patient. At least one embodiment may include an indicator that indicates a depletion of the energy source. In one or more embodiments, stimulation parameters may include one or more of amplitude, pulse width and frequency.

At least one embodiment of the invention may set stimulation parameters individually for each patient and may display an exchange indicator in the case of an implant, wherein the communications interface may be minimal as a result of a design of the implant.

One or more embodiments may include an active miniature implant, which may be provided without an antenna. In at least one embodiment, telemetry apparatuses may be omitted, such that the energy requirements are reduced and suitable antenna geometries would be omitted for the respective telemetry methods. In one or more embodiments, the implant may be used at greater tissue depths, for example as an endocardial implant. In at least one embodiment, such as with an endocardial implant, only telemetry methods that allow communication at greater tissue depths and that are accordingly complex may be used.

In at least one embodiment, in the event of activation by the trigger element, the control unit may trigger an electrical stimulation sequence including test stimulation pulses. In one or more embodiments, the control unit may receive a signal of the sensor, which corresponds to the trigger element. In at least one embodiment, the stimulation sequence may be preset.

In at least one embodiment of the invention, the control unit may vary the parameters of the test stimulation pulses with the electrical stimulation sequence. With a preset stimulation sequence, in one or more embodiments, the amplitude and/or the frequency of the test stimulation pulses may be reduced.

In at least one embodiment, when a lower threshold based on a previous parameter set of the test stimulation pulses is reached or undershot, the control unit may set a parameter set to deliver the therapeutic electrical stimulation pulses. As such, in one or more embodiments, a minimally effective value and a safety margin may be set. In one or more embodiments, determination of when the lower threshold value has been reached or undershot may be detected by auxiliary measurements. As such, in at least one embodiment, biophysical signals as a response to the test stimulation pulses and/or physical reactions may be monitored. In one or more embodiments, biophysical signals may, for example, include one or more of a surface electrocardiogram (ECG), an electroencephalogram, and an electromyogram. In at least one embodiment, physiological reactions may, for example, include one or more of muscle activity and perceptions of the patient.

In at least one embodiment, the control unit may receive and process a quitting signal at an end of the adaptation sequence in order to confirm the parameter set to deliver the therapeutic electrical stimulation pulses. In one or more embodiments, the control unit may predefine a safety setting of the stimulation parameters at the end of the adaptation sequence. As such, in at least one embodiment, the operational safety of the implant is improved.

At least one embodiment of the invention may include a processor that may estimate a service life of the energy source from a current parameter set to deliver the therapeutic electrical stimulation pulses and from an energy content of the energy source. As such, in one or more embodiments, the operational reliability of the implant is improved.

In at least one embodiment, the energy source may include a nuclear battery. One or more embodiments may determine the moment at which the nuclear battery is depleted, for example from the half-life of the nuclear battery and the calculated power consumption of the therapeutic electrical stimulation pulses.

In at least one embodiment, the trigger element may include a magnet. In one or more embodiments, the sensor may include a giant magnetoresistance sensor (GMR sensor). In at least one embodiment, the GMR sensors may respond to a magnetic field by exhibiting a sudden increase of their electrical resistance in the magnetic field.

According to at least one embodiment, the trigger element may cause a galvanic coupling-in of electrical pulses and/or a coupling-in of acoustic signals and/or a coupling-in of optical signals. For example, one or more embodiments of the invention may include a combinations of trigger elements and receiver elements in the implant, for example including one or more of a loudspeaker/microphone, a magnet/reed switch, a pulsed electromagnet/GMR sensor, an ultrasound transducer/ultrasound receiver, an LED/phototransistor, etc. In one or more embodiments, the trigger element may be individually selected as required.

In at least one embodiment, the implant may be a cardiac pacemaker, a defibrillator, an epicardial cardiac pacemaker, an electrodeless endocardial cardiac pacemaker, a neurostimulator or a muscle stimulator. In one or more embodiments, the implant may include a minimal unidirectional communications interface and may support a stimulation stimulus threshold test along with reprogramming of the stimulation parameters, and may include a battery exchange indicator.

One or more embodiments of the invention include a system including a medical implant that may be inserted into the human and/or animal body, and that may deliver electrical stimulation pulses to a bodily tissue. At least one embodiment may include a trigger element, which cooperates with an implant-side sensor, and a control unit coupled to the sensor. In one or more embodiments, the control unit may be activated depending on a sensor signal of the sensor in order to perform an adaptation sequence of stimulation parameters, wherein test stimulation pulses may be outputted by the control unit.

By way of at least one embodiment, the trigger element may include a magnet and the sensor may be a giant magnetoresistance sensor, of which the electrical resistance changes when the sensor approaches the magnet. In one or more embodiments, the trigger element may cause a galvanic coupling-in of electrical pulses and/or a coupling-in of acoustic signals and/or a coupling-in of optical signals.

At least one embodiment of the invention may include a method of setting a parameter set of therapeutic electrical stimulation pulses of an implant that may be inserted into the human and/or animal body. In one or more embodiments, a control unit may be activated by an external trigger element in order to perform an adaptation sequence of stimulation parameters, wherein test stimulation pulses may be outputted by the control unit. In at least one embodiment, the stimulus threshold test may be based on one or more of the evaluation of biophysical signals and/or physiological reactions and on the setting of the last amplitude assessed as being effective, and on a safety margin. One or more embodiments may include unidirectional communication without a telemetry antenna, such that the method may allow the pulse energy of an implant to be set individually for each patient. At least one embodiment may include an indicator that indicates the battery depletion of the implant.

In at least one embodiment, the test stimulation pulses may be varied until a lower threshold value is reached or undershot, and, when the lower threshold value is reached or undershot, a parameter set to deliver therapeutic electrical stimulation pulses may be set based on a previous parameter set of the test stimulation pulse.

In at least one embodiment, the adaptation sequence may be terminated by a quitting signal via the trigger element, or a safety setting may be activated with the absence of a quitting signal. As such, in one or more embodiments, the operational safety of the implant may be improved.

In at least one embodiment, a service life of the energy source may be estimated based on a parameter set of therapeutic electrical stimulation pulses and on an energy content of an implant-side energy source. As such, in one or more embodiments, the operational reliability of the implant may be improved.

### BRIEF DESCRIPTION OF THE DRAWINGS

The above and other aspects, features and advantages of at least one embodiment of the invention will be more apparent from the following more particular description thereof, presented in conjunction with the following drawings, wherein:

FIG. 1 shows a schematic illustration of a block diagram of an implant with an external trigger element;

FIG. 2 shows a schematic illustration of a section through an endocardial implant according to FIG. 1 with an external trigger element;

FIG. 3 shows a schematic illustration of a flow diagram of an adaptation sequence of stimulation pulses of an implant; and

FIG. 4 shows a schematic illustration of a schema to determine a service life of a nuclear battery; according to one or more embodiments of the invention.

### DETAILED DESCRIPTION OF THE INVENTION

The following description is of the best mode presently contemplated for carrying out at least one embodiment of the invention. This description is not to be taken in a limiting sense, but is made merely for the purpose of describing the general principles of the invention. The scope of the invention should be determined with reference to the claims.

In the drawings presented herein, functionally alike or similarly acting elements are denoted in each case by like reference signs. In addition, the drawings presented herein are schematic illustrations of the invention, and do not show specific parameters of the invention. Furthermore, the drawings presented herein merely reproduce one or more embodiments of the invention and are not intended to limit the invention to the illustrated embodiments.

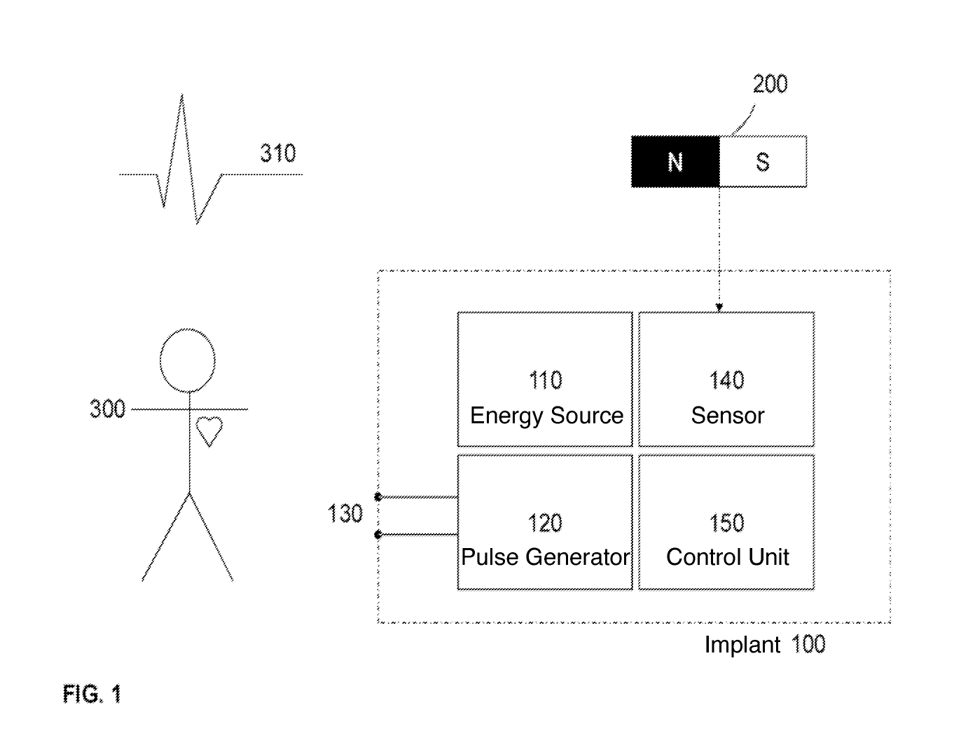

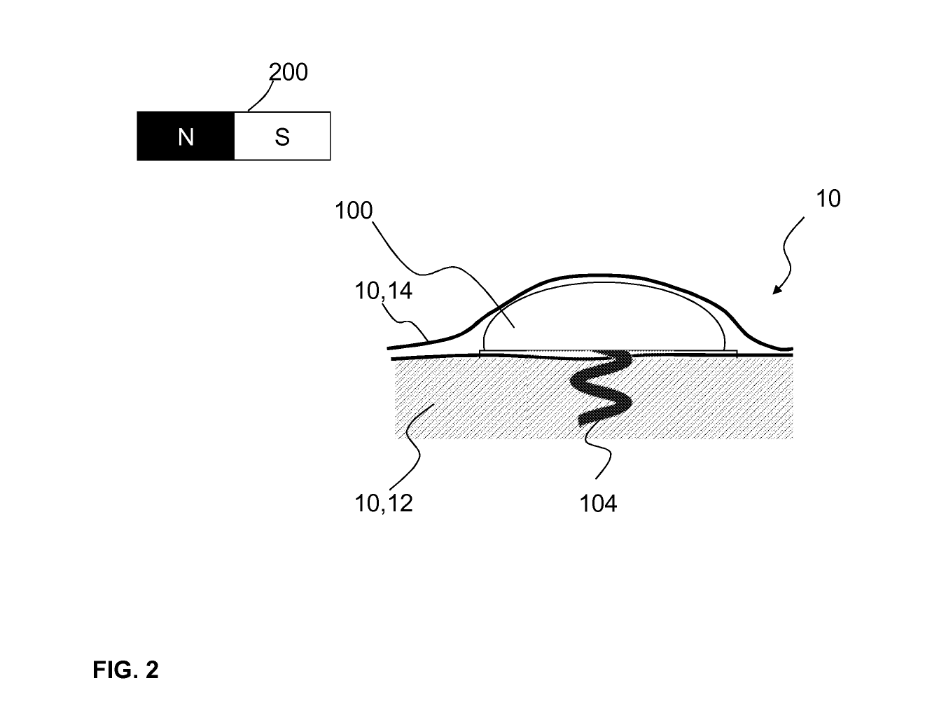

FIG. 1 shows a schematic illustration of a block diagram of an implant with an external trigger element, according to one or more embodiments of the invention. For example, FIG. 1 shows an arrangement with a block diagram of a medical implant 100 that may be inserted into the human and/or animal body, with an external trigger element 200 and a patient 300, according to at least one embodiment of the invention. In one or more embodiments, the implant 100 may deliver therapeutic stimulation pulses to the tissue. FIG. 2, in sectional view, shows a schematic illustration of an endocardial implant according to FIG. 1 with an external trigger element, according to one or more embodiments of the invention. As shown in FIG. 1, at least one embodiment may include a medical implant 100, such as an endocardial miniature implant 100, which may be used at an implantation site 10, for example between the myocardium 12 and pericardium 14. In one or more embodiments, the implant 100 may be fixed in the myocardium 12 using a fixing helix 104. In at least one embodiment, the implant 100 may require a minimization of the geometric dimensions and weight thereof. With the miniature implant, one or more embodiments may omit telemetry apparatuses, such that the energy requirements are reduced and suitable geometries for the respective telemetry methods may be omitted. With an endocardial implant, in at least one embodiment, only telemetry methods that allow a communication at great tissue depths may be used.

By way of one or more embodiments, the medical implant 100 may include one or more of, as shown in FIG. 1, an energy source 110, a pulse generator 120, a sensor 140, a control unit 150 and at least one electrode pair 130. In at least one embodiment, the energy source may be a nuclear battery, for example.

According to one or more embodiments, the control unit 150 may actuate the pulse generator 120 to deliver electrical pulses, which may include therapeutic stimulation pulses with normal operation. In at least one embodiment, the electrical pulses may be delivered via the electrode pair 130, formed as a dipole, to the myocardial tissue (as shown in FIG. 2) of a patient 300.

To set stimulation parameters individually for each patient, in one or more embodiments, the control unit 150 may be activated depending on a sensor signal of the sensor 140 in order to perform an adaptation sequence of stimulation parameters, in which test stimulation pulses may be outputted.

In at least one embodiment, the sensor 140 may identify the presence of a simple trigger element 200 via the patient 300 and may forward a corresponding sensor signal to the control unit 150. In one or more embodiments, the trigger element 200 may be a strong permanent magnet and the sensor 140 may be a GMR sensor. At least one embodiment, for example, may derive a surface ECG 310 of the patient 300 in order to identify the efficacy of the stimulation.

By way of one or more embodiments, the control unit 150 may, upon activation by the trigger element 200, trigger an electrical stimulation sequence with test stimulation pulses at the pulse generator 120. In at least one embodiment, an adaptation sequence of the stimulation parameters may be performed, with which a stimulus threshold may be detected, from which stimulation pulses are effective. In one or more embodiments, the efficacy of the test stimulation pulses may be identified with the surface ECG 310. In at least one embodiment, stimulation parameters may include amplitude and/or pulse width and/or frequency of the stimulation pulses.

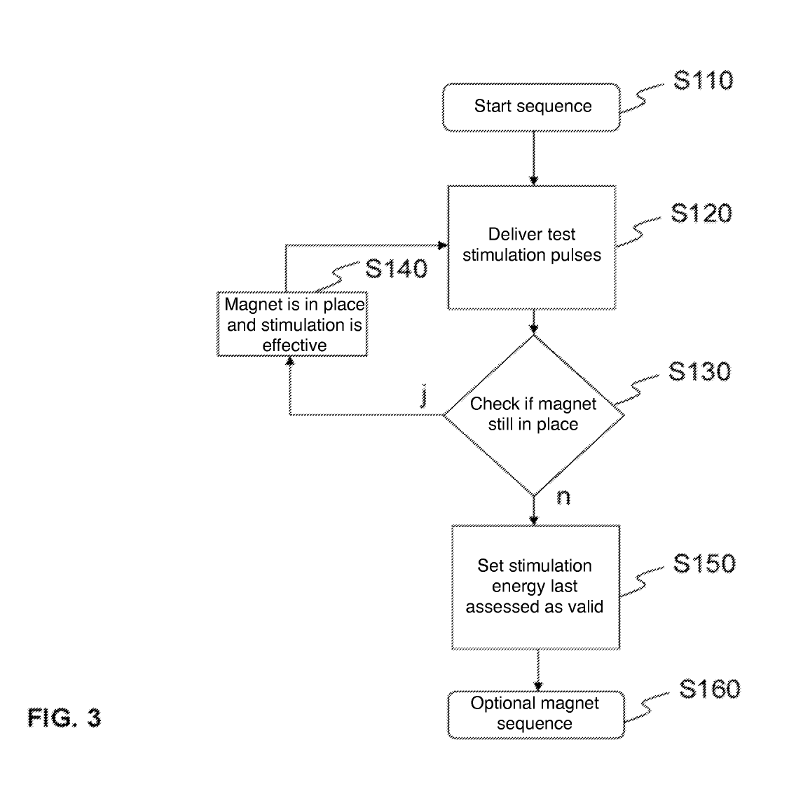

According to one or more embodiments, the course of the adaptation sequence to detect a stimulus threshold, which may be individual to each patient, with an amplitude adaptation of the test stimulation pulses is explained on the basis of a flow diagram as shown in FIG. 3.

As shown in FIG. 3, in at least one embodiment, the adaptation sequence may start by a defined introductory start sequence in step S110. In one or more embodiments, a start sequence may be, for example, a placement and removal of the magnet in the vicinity of the implant 100, which is communicated from the sensor 140 to the control unit 150 (shown in FIG. 1). Then, in at least one embodiment, the magnet may be placed back. In one or more embodiments, the adaptation sequence may be performed until the magnet is placed, for example until the trigger element 200 is identified by the sensor 140 after the start sequence. Whilst the adaptation sequence is being performed, by way of at least one embodiment, a predetermined number of test stimulation pulses with defined amplitude and pulse width may be delivered repeatedly in step S120. In step S130, one or more embodiments may check whether the magnet is still in place. If the magnet is still in place, depicted by “j” in step S130 in FIG. 3, in at least one embodiment, an indication is provided to the control unit 150 in the implant 100 that the user has assessed the current stimulation to be effective, such that the stimulation energy is reduced in a predefined manner for the following part of the adaptation sequence, at step 140. In one or more embodiments, the process may be repeated until the magnet is removed and the sensor 140 indicates to the control unit 150 that the stimulation is ineffective, depicted by “n” in step S130 in FIG. 3. In this case, in at least one embodiment, the stimulus threshold is reached, and the control unit 150 may terminate the adaptation sequence. At step 150, in one or more embodiments, the control unit 150, for the permanent therapeutic stimulation, may set the stimulation energy last assessed as valid, and may include a predefined safety margin.

Due to an optional magnet sequence in step S160, by way of at least one embodiment, the user may quit this setting with a quitting signal and may confirm such an action in the control unit 150, otherwise a safety setting may be activated.

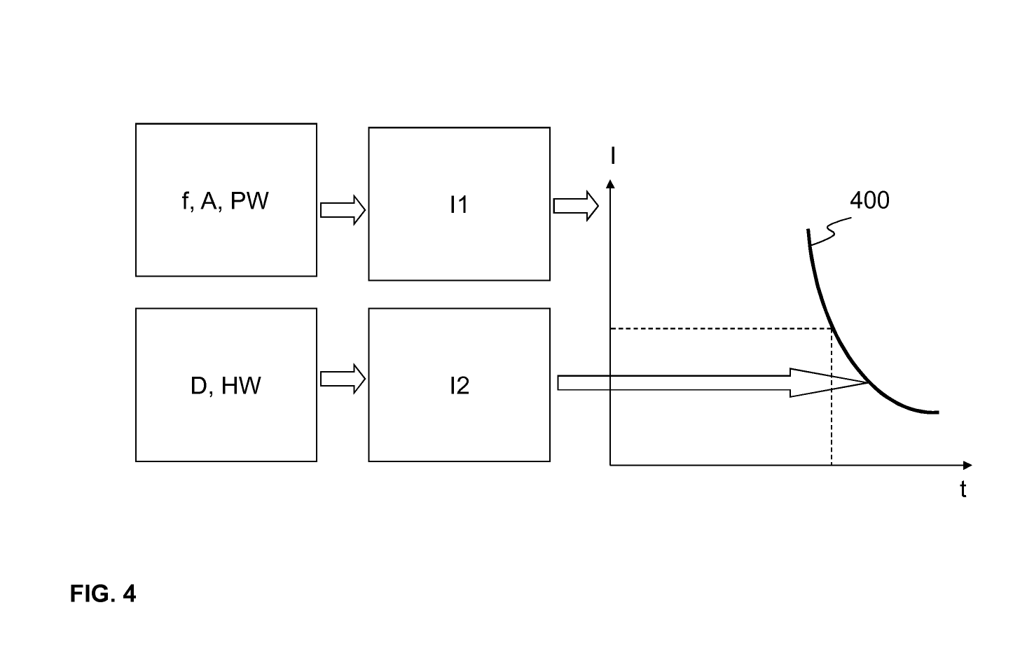

FIG. 4 shows a schematic illustration of a schema to determine a service life of a nuclear battery, which may be used as an energy source 110 (shown in FIG. 1), according to one or more embodiments of the invention. In at least one embodiment, the control unit 150 may provide the information concerning the selected stimulation amplitude, in which the selection of the stimulation amplitude may be selected in accordance with a fixed schema. Using an external ECG writer, in at least one embodiment, the signals may then be recorded and the stimulation amplitudes may be counted. In one or more embodiments, the remaining residual running time may then be read out from a table (for example as part of a manual or on a web page associated with the ECG writer), under assumption of the current stimulation amplitude, in combination with the battery start date and/or the production date of the energy source 110, for example a nuclear battery.

By way of at least one embodiment, the assessment of the remaining operating time with use of the nuclear battery may be based on the predetermined stimulation parameters set via the adaptation sequence in FIG. 3. For example, in one or more embodiments, the remaining operating time may be based on a frequency f, an amplitude A and pulse width PW, which may be recalculated into a maximum current consumption I1 of the implant 100. According to at least one embodiment, since a nuclear battery may deliver a maximum current independently of the removed current, the maximum current being dependent only on the half-life (HW) of a radionuclide used, depicted by curve 400 in FIG. 4, a maximum deliverable current 12 over time may be calculated from the half-life time (HW) and from the age (D) of the nuclear battery. In one or more embodiments, the remaining operating time may be read off on the basis of such data. In at least one embodiment, when the service life of the implant 100 is approached, the control unit 150 may deliver a corresponding indicator signal at the maximum service life, for example with a resetting of stimulation parameters.

Embodiments of the invention may provide a small implantable pulse generator with a stimulus threshold setting and an exchange indicator, without a telemetry function.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A medical implant configured to be inserted into a human and/or animal body, comprising:
an energy source;
a pulse generator;
a control unit;
at least one electrode pair that delivers therapeutic electrical stimulation pulses to a bodily tissue; and,
a sensor that identifies an implant-external trigger element;
wherein the sensor is provided on an implant side of the medical implant,
wherein the control unit is coupled to the sensor, and,
wherein the control unit is activated depending on a sensor signal of the sensor in order to perform an adaptation sequence of stimulation parameters of test stimulation pulses, such that said test stimulation pulses are outputted by the control unit.

2. The medical implant as claimed in claim 1, wherein during an event of activation by the implant-external trigger element, the control unit is configured to trigger an electrical stimulation sequence comprising said test stimulation pulses.

3. The medical implant as claimed in claim 2, wherein the control unit is further configured to vary the stimulation parameters of the test stimulation pulses with the electrical stimulation sequence.

4. The medical implant as claimed in claim 1, wherein when a lower threshold based on a previous parameter set of the test stimulation pulses is reached or undershot, the control unit is configured to set a parameter set to deliver the therapeutic electrical stimulation pulses.

5. The medical implant as claimed in claim 4, wherein the control unit is further configured to receive and to process a quitting signal at an end of the adaptation sequence of stimulation parameters to confirm the parameter set to deliver the therapeutic electrical stimulation pulses.

6. The medical implant as claimed in claim 4, wherein the control unit is further configured to predefine a safety setting of the stimulation parameters at an end of the adaptation sequence of stimulation parameters.

7. The medical implant as claimed in claim 1, further comprising a processor configured to estimate a service life of the energy source from a current parameter set to deliver the therapeutic electrical stimulation pulses and from an energy content of the energy source.

8. The medical implant as claimed in claim 1, wherein the energy source comprises a nuclear battery.

9. The medical implant as claimed in claim 1, wherein the medical implant comprises a cardiac pacemaker, a defibrillator, an epicardial cardiac pacemaker, an electrodeless endocardial cardiac pacemaker, a neurostimulator, or a muscle stimulator.

10. A system configured to be inserted into a human and/or animal body and to deliver electrical stimulation pulses to a bodily tissue, wherein the system comprises:
a medical implant comprising
an energy source,
a pulse generator,
a control unit,
at least one electrode pair that deliver the therapeutic electrical stimulation pulses to the bodily tissue, and,
a sensor; and,
a trigger element;
wherein the trigger element cooperates with the sensor,
wherein the sensor identifies the trigger element;
wherein the sensor is provided on an implant side of the medical implant,
wherein the control unit is coupled to the sensor, and,
wherein the control unit is activated depending on a sensor signal of the sensor in order to perform an adaptation sequence of stimulation parameters of test stimulation pulses, such that said test stimulation pulses are outputted by the control unit.

11. The system as claimed in claim 10, wherein the trigger element comprises a magnet and the sensor is a giant magnetoresistance sensor.

12. The system as claimed in claim 10, wherein the trigger element causes one or more of a galvanic coupling-in of electrical pulses, a coupling-in of acoustic signals and a coupling-in of optical signals.

13. A method of setting a parameter set of therapeutic electrical stimulation pulses of a medical implant configured to be inserted into a human and/or animal body, comprising:
providing a medical implant, wherein the medical implant comprises
an energy source;
a pulse generator;
a control unit;
at least one electrode pair that delivers electrical stimulation pulses to a bodily tissue; and,
a sensor that identifies an implant-external trigger element,
wherein the sensor is provided on an implant side of the medical implant,
wherein the control unit is coupled to the sensor, and,
wherein the control unit is activated depending on a sensor signal of the sensor;
activating the control unit by said implant-external external trigger element, such that the control unit performs an adaptation sequence of stimulation parameters of test stimulation pulses, and such that said control unit outputs said test stimulation pulses.

14. The method as claimed in claim 13, wherein the test stimulation pulses are varied until a lower threshold value is reached or undershot, and when the lower threshold value is reached or undershot, a parameter set to deliver the therapeutic electrical stimulation pulses is set based on a previous parameter set of the test stimulation pulses.

15. The method as claimed in claim 13, wherein the adaptation sequence of stimulation parameters is terminated by a quitting signal via the implant-external trigger element, or wherein a safety setting is activated with an absence of a quitting signal.

16. The method as claimed in claim 13, wherein a service life of the energy source is estimated based on a parameter set of therapeutic electrical stimulation pulses and based on an energy content of an implant-side energy source.
